(19) United States
(12) Patent Application Publication
(10) Pub. No.: US 2006/0084924 A1
(43) Pub. Date: Apr. 20, 2006

(54) Insulin pump pouch assembly

(76) Inventor: Susi Koch, Huntington Beach, CA (US)

Correspondence Address: Karl M. Steins;Suite 120, 2333 Camino del Rio South, San Diego, CA, 92108, US

(21) Appl. No.: 10/970,747

(22) Filed: Oct. 20, 2004

(51) Int. Cl. A61M 1/00; A61M 5/32

(52) U.S. Cl. 604/179; 604/151

(57) ABSTRACT

An Insulin Pump Pouch Assembly is disclosed The pouch is sized to receive a conventional personal insulin pump. The pouch provides not only physical protection for the pump, but also provides an aesthetically-pleasing container. One particularly important aspect is that the dispense tube is protected both physically and from view to improve aesthetics. The pouch is further attachable by belt or clip.

### BACKGROUND OF THE INVENTION

1. Field of the Invention

This invention relates generally to portable drug delivery systems and, more specifically, to an Insulin Pump Pouch Assembly.

2. Description of Related Art

Individuals suffering from diabetes often must receive regular injections of insulin. Somewhat recently, a less-intrusive method of receiving insulin treatments has become available—the portable insulin pump. FIG. 1 is a perspective view of a conventional portable insulin pump/dispensing system 10. The system 10 comprises a pump apparatus 12 having a control panel 20 to allow the user to schedule the dosages and/or to demand a manual dose. The insulin “injection” travels down thin tubing 18 that leads from the pump apparatus 12 to the users body at the infusion site (usually located at the patient's stomach, buttocks or thigh). At the end of the tubing 18, is the dispensing head 14, which attaches to the patient's body with an adhesive-coated patch 16.

While evolution of the portable insulin dispensing system 10 provides the user with flexibility and comfort never before experienced by diabetics, there are some drawbacks. First, the user must have constant access to the control panel 20 in the event that adjustment is necessary—the problem is that the tubing 18 must lead into the person's clothing so that it can contact the skin. Since the tubing 18 leads from outside to inside of the patient's clothes, it is very hard to disguise, and can tend to make the patient self-conscious. Second, the tubing 18 is fairly fine in gauge; it is very easy to get is hung up on things during the course of the patient's day—this can result in the patch 16 actually being pulled off of the patient's skin, sometimes without their knowledge.

What is needed, then, is a device for holding the pump apparatus 12 external to the patient's body to hide the tubing 18 and further prevent the tubing 18 from getting snagged.

### SUMMARY OF THE INVENTION

In light of the aforementioned problems associated with the prior systems and devices, it is an object of the present invention to provide an Insulin Pump Pouch Assembly. The pouch should be sized to receive a conventional personal insulin pump. The pouch should provide not only physical protection for the pump, but should also provide an aesthetically-pleasing container. One particularly important aspect is that the dispense tube be protected both physically and from view to improve aesthetics. The pouch should be attachable by belt or clip.

### BRIEF DESCRIPTION OF THE DRAWINGS

The objects and features of the present invention, which are believed to be novel, are set forth with particularity in the appended claims. The present invention, both as to its organization and manner of operation, together with further objects and advantages, may best be understood by reference to the following description, taken in connection with the accompanying drawings, of which:

FIG. 1 is a perspective view of a conventional personal insulin dispensing system;

FIG. 2 is a front perspective view of a preferred embodiment of the insulin pump pouch assembly of the present invention;

FIG. 3 is another front perspective view of the assembly of FIG. 2; and

FIG. 4 is a partial rear perspective view of the assembly of FIGS. 2 and 3.

### DETAILED DESCRIPTION OF THE PREFERRED EMBODIMENTS

The following description is provided to enable any person skilled in the art to make and use the invention and sets forth the best modes contemplated by the inventor of carrying out her invention. Various modifications, however, will remain readily apparent to those skilled in the art, since the generic principles of the present invention have been defined herein specifically to provide an Insulin Pump Pouch Assembly.

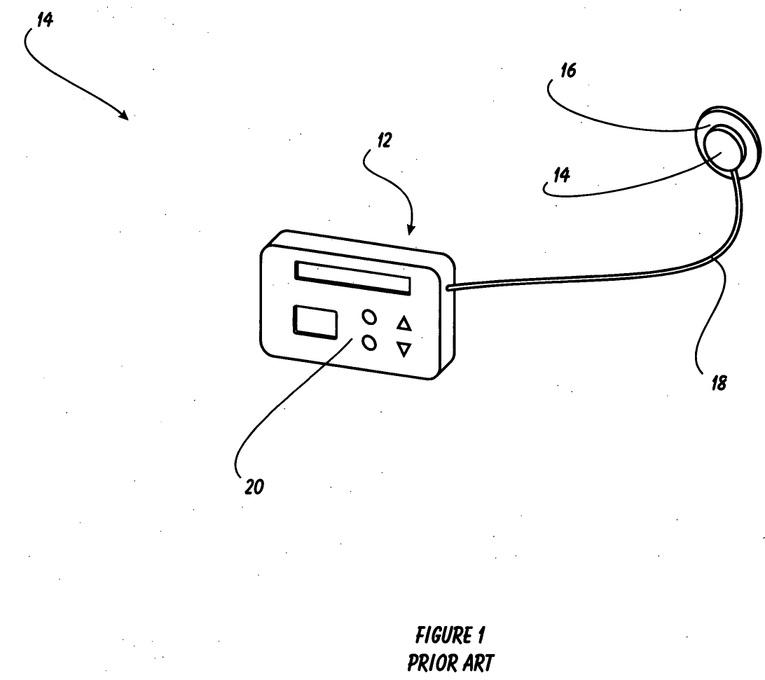

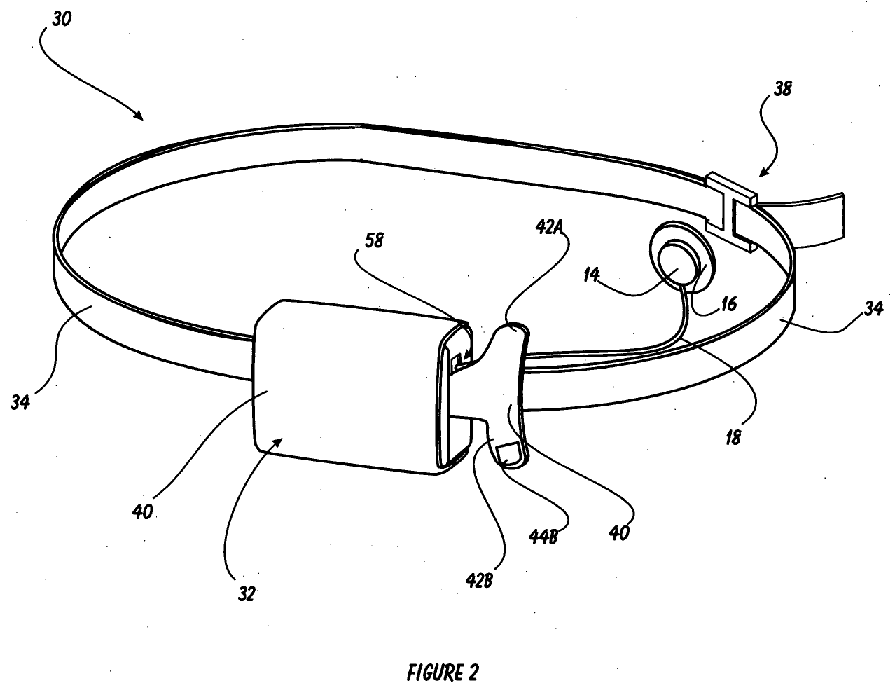

The present invention can best be understood by initial consideration of FIG. 2. FIG. 2 is a front perspective view of a preferred embodiment of the insulin pump pouch assembly 30 of the present invention. The assembly 30 comprises a pump pouch 32 for holding the insulin pump, and a belt 34 (or clip) for attaching the assembly 30 to the patient's body. The pouch 30 is sized to accept the pump apparatus (see FIG. 1) within it. There is further an aperture 58 in the side of the pouch 32 where the tubing 18 can protrude.

The assembly 30 provides protection for the tubing 18 in the form of the tubing protector 40. The tubing protector 40 extends from the side of the pouch 32 and terminates in a first flap 42A and second flap 42B. The flaps 42 are configured to wrap around the tubing 18 and belt 34 closely adjacent to the pouch and then attach to one another on the other side of the belt 34. The result is that the tubing 18 is held securely close to the patient's body to prevent it snagging. Furthermore, the tubing 18 is hidden from view in order to eliminate potential embarrassment for the patient. The fastener flap 44A at the end of the first flap 42A and its counter-part at the end of the second flap 42B are preferably the two parts of a hook-and-loop type of fastener, which secures the tubing 18.

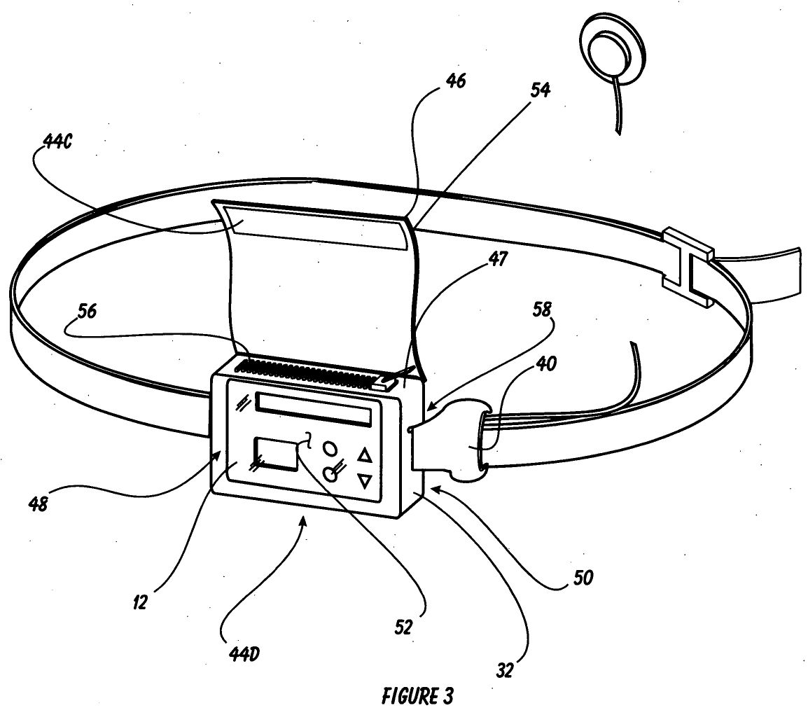

The pouch 32 has a cover flap 40 that can be closed over the face of the pouch 32, for decorative and protective purposes. The pouch 32 and its related components will typically be constructed from a washable cloth material that provides a durable, yet aesthetically pleasing appearance. Versions may be available that incorporate patient identity or other indicia inscribed thereon, as well as having portions made from or covered with decorative cloth. Now turning to FIG. 3, we can continue to examine the present invention.

FIG. 3 is another front perspective view of the assembly 30 of FIG. 2. In this view, the cover flap 46 has been lifted to expose the top face 47 of the pouch 32. The top face 47 has a pump access opening 54 formed in it to enable the pump apparatus 12 to be placed in, or removed from, the pouch 32. The opening 54 will be held closed by a zipper 56 or other conventional closure device.

The cover flap 46 has a fastener pad 44C at its distal edge that cooperates with another fastener pad 44D located on the bottom face of the pouch to hold the cover flap 46 closed tight against the front face 48 of the pouch 32 (as depicted in FIG. 2). The pouch 32 has a window 52 on its front face 48, through which the control panel of the pump apparatus 12 is viewable, and through which the touch controls on the control panel are operable. By closing the cover flap 46, the user disguises the purpose of the pouch 32, and further prevents the inadvertent operation of buttons on the control panel of the pump apparatus 12.

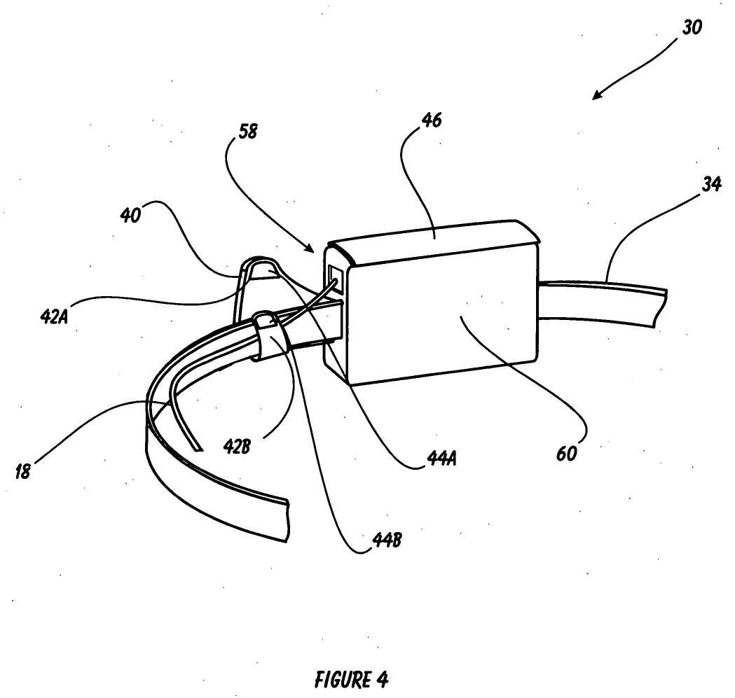

The tubing protector 40 is depicted here with its two flaps attached to one another wrapped around the tubing 18 and the belt 34. As can be seen, the tubing 18 is shielded from view by the tubing protector. If we finally turn to FIG. 4, we can examine the final details of this novel invention.

FIG. 4 is a partial rear perspective view of the assembly 30 of FIGS. 2 and 3. This view depicts the pouch 32 with the cover flap 46 in the closed position. The tubing protector 40 is partially open in order to expose the fastener pads 44A and 44B at the tips of the flaps 42A and 42B.

When attached to a person's body, the tubing 18 may be fed on the inside of the belt 34 until it reaches the entry point in the person's clothing. The rear face 60 will be held tight to the waist of the wearer, and will appear to be a small “fanny pack,” rather than a medical device. The belt 34 may be fixedly attached to the pouch 32, as shown here, or it might pass through a loop or loops formed on the rear face 60 of the pouch 32 to permit easy adjustment of the location of the pouch 32 in relation to the buckles.

Those skilled in the art will appreciate that various adaptations and modifications of the just-described preferred embodiment can be configured without departing from the scope and spirit of the invention. Therefore, it is to be understood that, within the scope of the appended claims, the invention may be practiced other than as specifically described herein.

What is claimed is:

1. An insulin pump pouch assembly comprising:
a belt of adjustable length configurable into a loop at least 12 inches in diameter;
a pouch, comprising:
a front face;
a pair of opposing side faces adjacent to said front face;
a bottom face adjacent to said side faces and said front face; and
a top face opposite said bottom face,. further defined by an opening formed therein.

2. The assembly of claim 1, further comprising a tubing protector extending from one said side face.

3. The assembly of claim 2, wherein said front face is further defined by a window formed therein, said window being substantially transparent.

4. The assembly of claim 2, wherein one said side face comprises an aperture formed therein.

5. The assembly of claim 2, wherein said tubing protector generally defines a “T” shape constructed from a flexible material.

6. The assembly of claim 5, wherein said tubing protector comprises a proximate end attached to said pouch and a distal end opposite said proximate end, said distal end defined by a pair of flaps extending therefrom.

7. The assembly of claim 6, wherein said flaps define a front surface and a rear surface, one said flap further comprising a fastener pad attached to its said front surface and the other said flap comprising a fastener pad attached to its said rear surface.

8. The assembly of claim 7, wherein said pouch is further defined by a zippered closure at said top face opening.

9. The assembly of claim 8, further comprising a cover flap extending from said top face to said bottom face, and detachably attachable to said bottom face.

10. The assembly of claim 9, wherein said cover flap is defined by a fastener pad at said bottom-face attachable end and said bottom face is further defined by a fastener pad attached thereto.

11. An insulin pump and pouch assembly combination, comprising:
an insulin pump having a pump apparatus defined by a control panel and tubing extending from said pump apparatus;
a pouch assembly comprising:
a belt of adjustable length configurable into a loop at least 12 inches in diameter;
a pouch, comprising:
a front face;
a pair of opposing side faces adjacent to said front face;
a bottom face adjacent to said side faces and said front face;
a top face opposite said bottom face, further defined by an opening formed therein; and a rear face; and
wherein said pouch is configured to accept said pump apparatus therewithin through said opening.

12. The combination of claim 11, wherein said front face is further defined by a window formed therein, said window being substantially transparent whereby said control panel is viewable therethrough.

13. The combination of claim 12, further comprising a tubing protector extending from one said side face.

14. The combination of claim 13, wherein one said side face comprises an aperture formed therein allowing said tubing to exit said pouch.

15. The combination of claim 14, wherein said tubing protector generally defines a “T” shape constructed from a flexible material.

16. The combination of claim 15, wherein said tubing protector comprises a proximate end attached to said pouch and a distal end opposite said proximate end, said distal end defined by a pair of flaps extending therefrom.

17. The combination of claim 16, wherein said flaps define a front surface and a rear surface, one said flap further comprising a fastener pad attached to its said front surface and the other said flap comprising a fastener pad attached to its said rear surface.

18. The combination of claim 17, wherein said tubing protector flap fastener pads attachable to one another to form a loop around said tubing and said belt.

19. An insulin pump pouch assembly comprising:
a pouch, comprising:
a front face;
a rear face
a pair of opposing side faces adjacent to said front face;
a bottom face adjacent to said side faces and said front face; and
a top face opposite said bottom face, further defined by an opening formed therein; and
a belt clip extending from said rear face.
